(19) United States
(12) Patent Application Publication
(10) Pub. No.: US 2013/0041439 A1
(43) Pub. Date: Feb. 14, 2013

(54) Cold cap

(71) Applicant: James Joseph Gallagher, New Carrollton, MD (US)

(72) Inventor: James Joseph Gallagher, New Carrollton, MD (US)

(21) Appl. No.: 13/137,411

(22) Filed: Aug. 12, 2011

(51) Int. Cl. A61F 7/10

(52) U.S. Cl. 607/109

(57) ABSTRACT

The purpose of this invention is to extend life. When a person's heart stops beating through any number of causes, the lack of oxygen carried by the blood to the brain occurs, causing the condition of suspended animation. The deterioration of the brain cells will result and the spark of life is forever extinguished. The Cold Cap is intended to extend the time between the end of suspended animation and the beginning of the deterioration of the brain cells.

### OBJECT OF THIS INVENTION

The following invention is designed, by the use of the Cold Cap, to slow the degeneration of the brain cells because of the interruption of oxygen being supplied to the brain by the blood circulatory system, specifically when the heart stops beating. This Cold Cap prolongs the life of an injured victim when the heart stops beating in order to perform life saving techniques as needed in cases of drowning, loss of blood, electric shock, heart attack and the like. The device can be employed to extend the period between the onset of the reduction or stoppage of oxygen to the brain and the commencement of irreversible damage to the brain. The device will temporarily prolong life, improve the odds for minimizing brain damage and insuring the survival until means of administering conventional lifesaving techniques can be employed.

The device, a Cold Cap, would be used to extend the period of suspended animation from the time the oxygen supplied to the brain through blood circulation stops and the commencement of the deterioration of the brain cells begins.

The Cold Cap would be used in emergencies when the heart stops beating, thus creating a loss of circulation of the blood throughout the body, causing lack of oxygen to the brain, usually causing brain death and ultimately death to the body itself.

The Cold Cap would be used to temporarily prolong the life of the victim in order to provide a better chance of survival by administering life saving techniques to save the victim.

It is known that the circulatory blood system carries blood and oxygen to the brain. They say that any interruption of the oxygen supply quickly initiates deterioration of brain cells and, ultimately, causes death. As has been proven by such experience as that cited in the Cold Cap Device Brief, as well as recent medical and scientific investigation, if the temperature of brain cells can be quickly reduced, the onset of irreversible deterioration of the brain can be postponed until techniques can be applied.

It has been acknowledged that when frigid conditions are present lives were saved which would otherwise be lost.

As far back as 1780, history includes events where hundreds of victims who had drowned in the cold water of Dublin Ireland's Anna Liffey River were revived. More recently, a young woman jogging in New York City's Central Park was attacked and beaten by thugs who left her for dead. Later, the doctors who examined her condition declared that because her head had ended up in the cold mud, her life was saved. There was a case when a pre-teen youth fell into Lake Michigan and was submerged for close to an hour and was later revived. There are many such documented cases.

There have been times that, through human ignorance, lives have been lost. When the Air Florida plane crashed at the Fourteenth Street Bridge over the Potomac River in D.C. many victims could have been saved if the rescuers had realized that (like the boy who had fallen into Lake Michigan) some of the submerged passengers in that plane could have been saved. A number of the victims of the US Air flight that went off the end of the runway into the water at La Guardia Airport, in frigid weather could also have have been saved. The cold weather in both cases would have had the same effect as the Cold Cap.

These are some of the events that happened to me personally:
A. A friend of mine had a heart attack while we were at a dance. Some nurses at the next table jumped right in and tried to save him, but to no avail. As I stood there, I noticed that he was sweating heavily, and if they had packed him in ice or if they had a Cold Cap it could have helped.
B. At a neighbor's back yard swimming pool, a young boy of three was being watched by his grandmother. She fell asleep, and when she awakened, she found the boy at the bottom of the pool. She called for help and when the boy was removed from the pool, revival attempts ended with the boy dying after ten months in a coma. If the Cold Cap had been used, it might have saved the child.
C. A 63 year old in-law of mine was cutting his lawn on a day when the temperature was in the upper 80's. He passed out, and his wife saw him fall. She ran out and tried to help. She called the next door neighbors who called the Rescue Squad. They arrived in about ten minutes, and they tried to revive him. He lasted a week. The doctor told his daughter that if they could have revived him sooner, he would have had a chance. If the Cold Cap had been available at that time, all three may have been saved.

The device is comprised of a head enclosing system that includes a means to contain and apply a temperature reducing medium to the brain with sufficient rapidity so that the temperature of the brain and associated blood supplying systems can be reduced to temperature levels sufficient to inhibit blood cell deterioration for a suitable length of time.

The head enclosing system consists of a cap-like multi-layered garment composed of inner and outer flexible, fluid-impervious material walls formed to completely enclose the head and through continuous vertical connections between the inner and the outer walls are vertical cells each capable of holding a cooling liquid. Means are also provided to allow input of oxygen into the device as may be warranted. This is provided by means of an opening in the front of the Cold Cap at the nose/mouth position, over which a removable connection of an oxygen mask will be attached to the front of the Cold Cap.

This opening would also be used to slide the cap over the head of the victim before the mask is installed. The removable connection for an oxygen mask will be attached to the front of the Cold Cap.

Cooling can be provided in various ways. Appropriately positioned refrigerators can be utilized to keep the contained liquid at a proper temperature pending need on ambulances, fire trucks, or other emergency vehicles. Multiple liquids which reduce temperatures when combined, or compressed gases that do the same when expanded are other sources of requisite cooling, eliminating the need for separate refrigeration.

It is known that the circulatory blood system carries blood and oxygen to the brain. Any interruption of oxygen supply quickly initiates deterioration of the brain cells causing the victim to expire. By keeping the head cold, the brain damage resulting in death can be delayed. As has been proven by such experience as that cited in this Cold Cap Brief, as well as recent medical and scientific investigation, if the temperature of the brain cells can be quickly reduced, the onset of irreversible deterioration of the brain can be postponed, and this delay could allow time for life saving techniques to be implemented. The head covering device, the Cold Cap, is designed to keep the head cold. It is my belief that the longer the spark of life is maintained, the better the chance the victim has to survive.

### Cold Cap; Problem and Prior Art

to my knowledge, the only prior art to this invention is the possible use of an ice bag.

This cap would be stored in a freezer ready to use. The detachable oxygen mask would be attached by Velcro to the Cold Cap when stored.

The foregoing purpose, features and advantages of this invention is attained by the method of using frozen but not rigid liquid.

The beauty of this invention is that it can be manufactured in large quantities ready for use until needed. This cap is designed to be placed over the head of the victim immediately after the cap's removal from the freezer. Please note that time is of the essence.

As most of the accidental experience with prior brain-cooling phenomena has involved bodies of cold water and the like, the prior art of that kind has involved existing bodies of cold water that served as both cause of need and means of delivering the required temperature reduction. In medical or surgical instances the prior art was the use of ice bags etc. to deliver temperature reducing means readily available in a hospital situation.

### DRAWINGS OF COLD CAP, FIGS. 1-11

FIG. 1: Front Elevation of Cold Cap with Oxygen Mask on

FIG. 2: Front Elevation of Cold Cap with Neck Strap open

FIG. 3: Front Elevation of Cold Cap with Neck Strap open and folded over showing band of Velcro on outside to receive Oxygen Mask [NOTE: Wherever Velcro is mentioned for attachment, other methods, such as snaps for example, may be used.]

FIG. 4: Back Elevation of Oxygen Mask with Velcro around perimeter to be mounted on Velcro around nose/mouth opening

FIG. 5: Front Elevation of Cold Cap with Oxygen Mask off

FIG. 6: Side Elevation of Cold Cap with Oxygen Mask on

FIG. 7: Horizontal Cross-Section A-A

FIG. 8: Vertical Cross-Section B-B

FIG. 9: Isometric of back of Oxygen Mask with Velcro around the perimeter

FIG. 10: Isometric of front and side of Oxygen Mask ready to fit onto Velcro around Nose/Mouth opening in front portion of Cold Cap

FIG. 11: Isometric of Cold Cap with Nose/Mouth opening with Velcro around the perimeter to receive Oxygen Mask

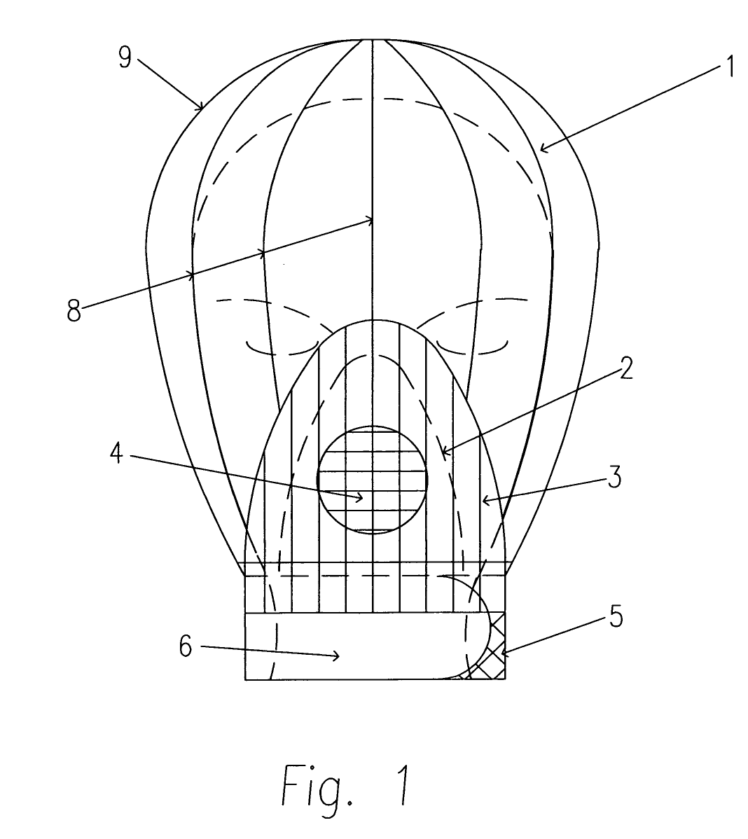

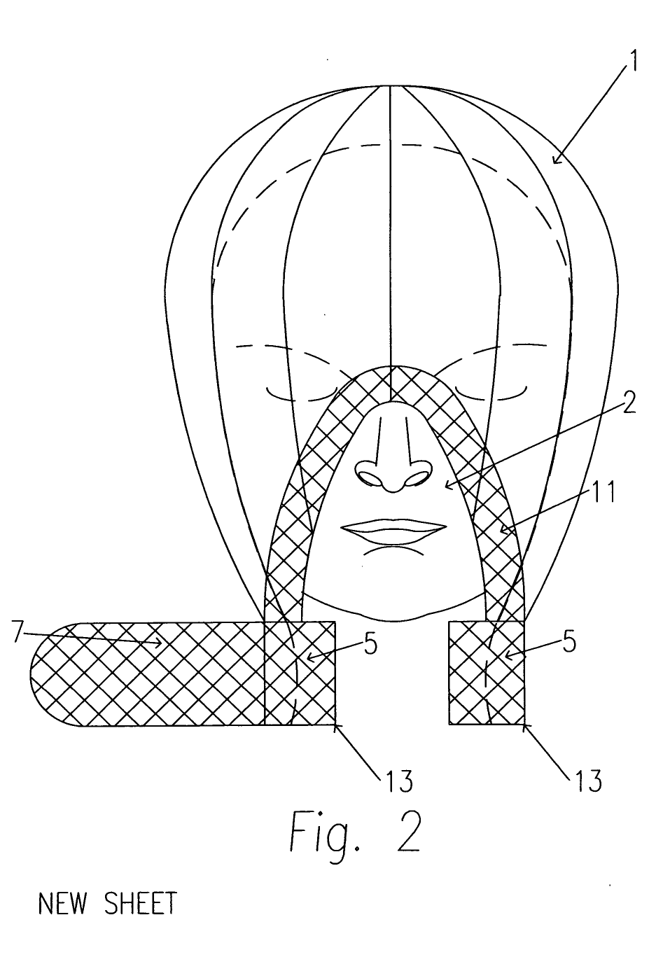

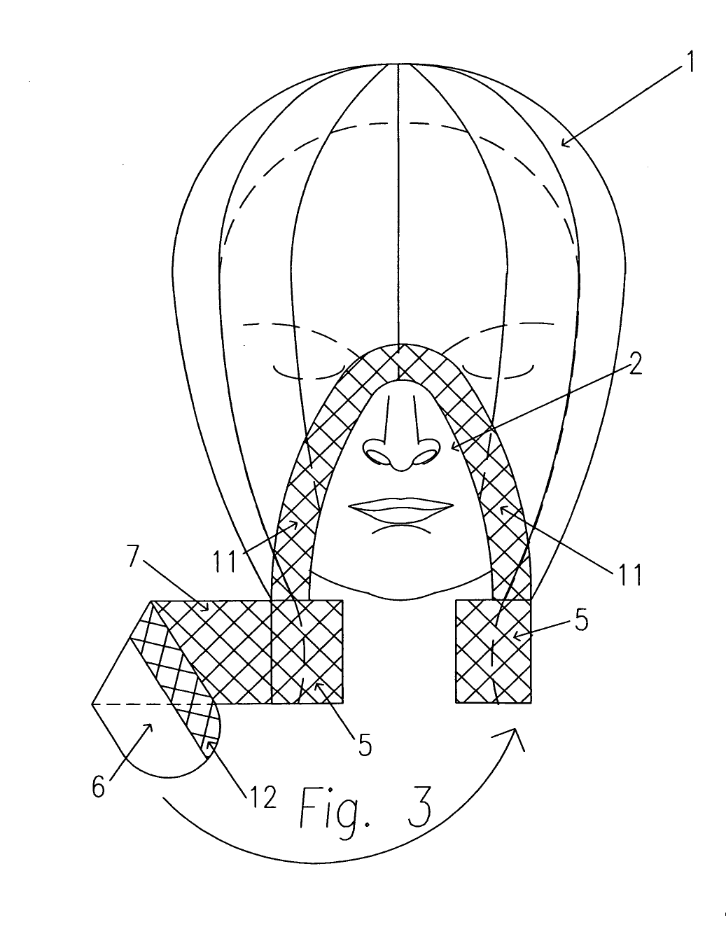

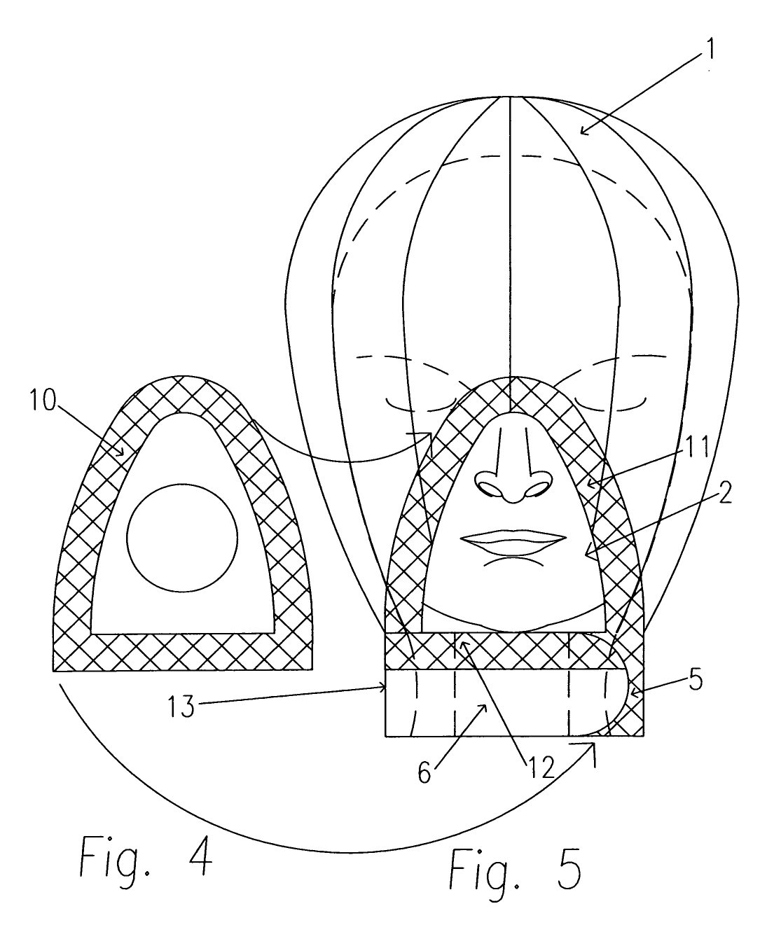

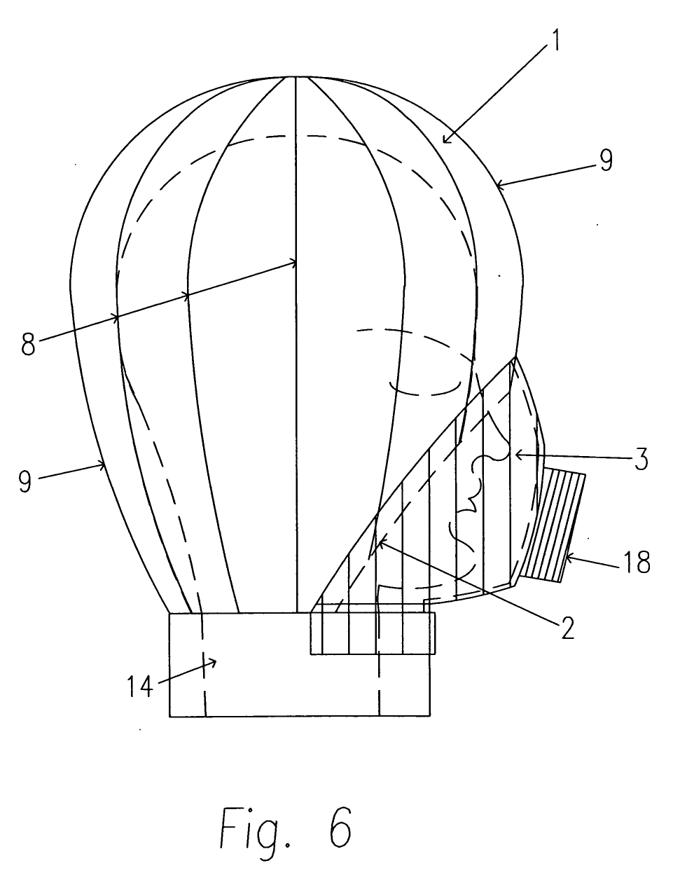

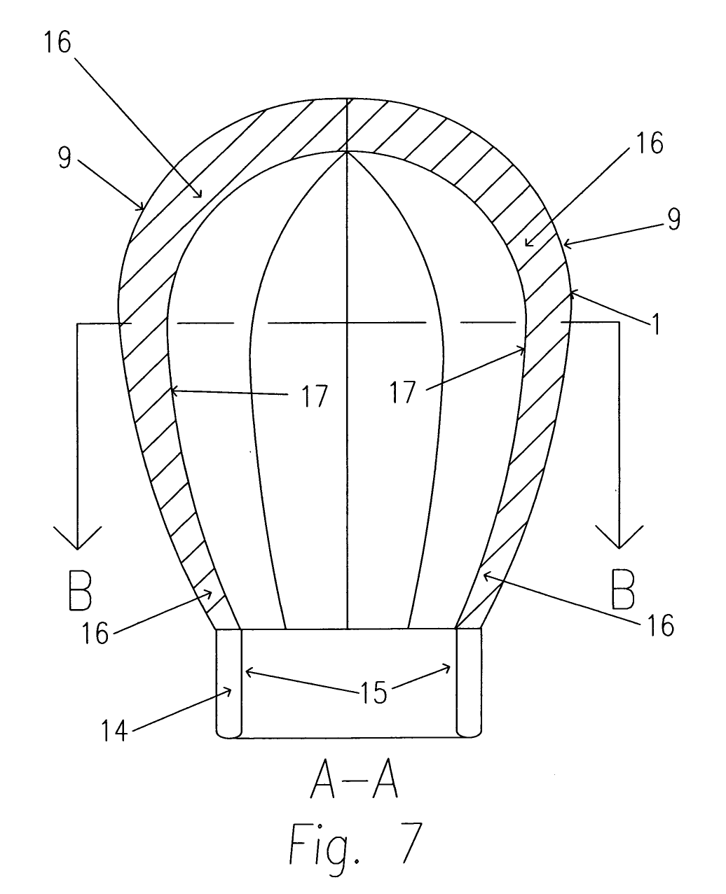

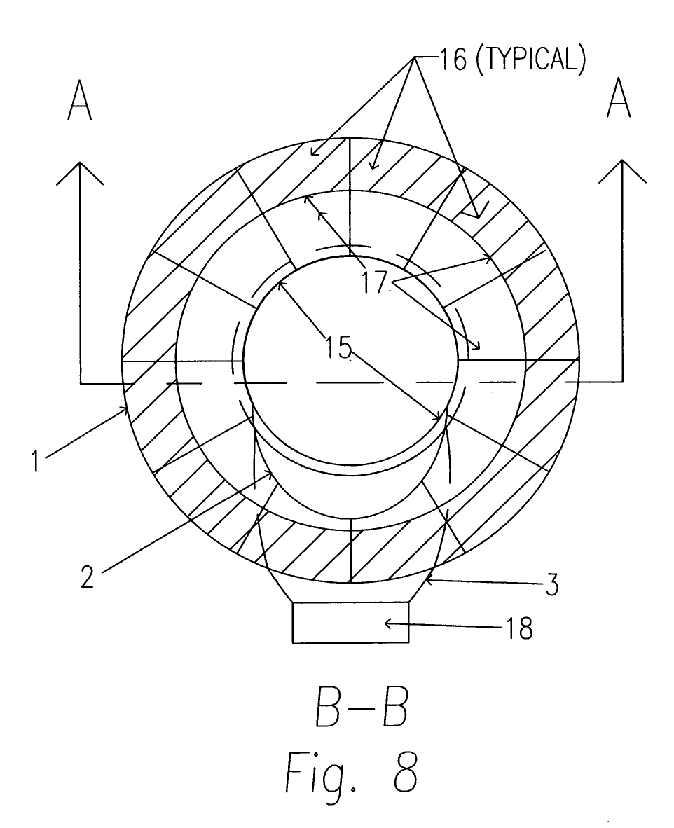

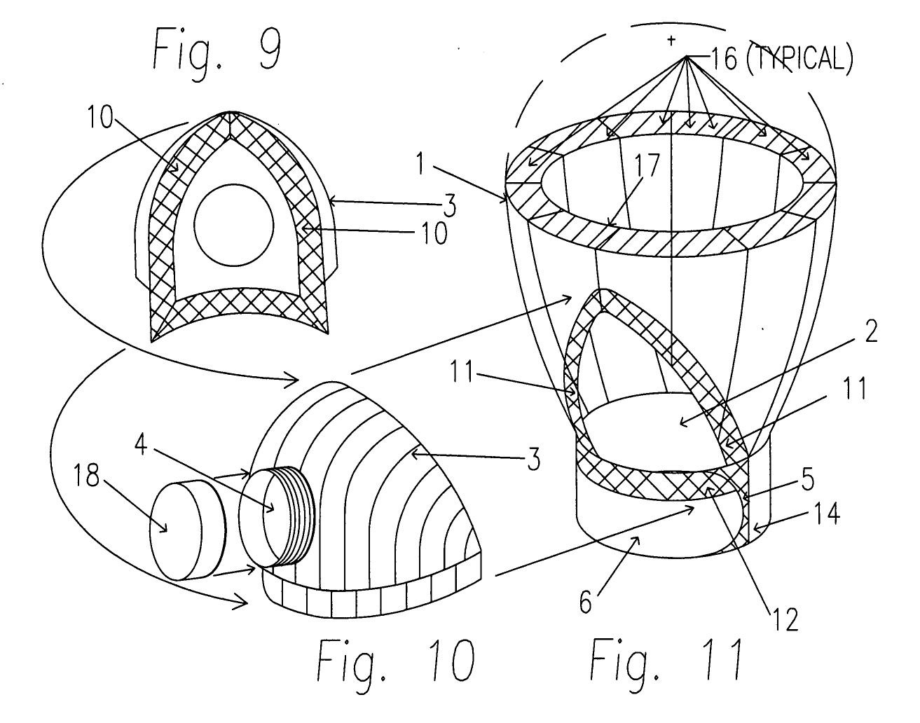

### DRAWING NOTES 1 THROUGH 18

1. Cold Cap

2. Nose/Mouth opening in Cold Cap to receive Oxygen Mask

3. Oxygen Mask

4. Oxygen Hose Connection with a screw-on cap

5. Velcro applied to outside front of neck collar

6. Outside of front neck flap

7. Inside of front neck flap covered with Velcro

8. Latex-like dividers between Coolant Cells, typical

9. Outer latex-like layer

10. Velcro applied to back perimeter of Oxygen Mask

11. Velcro applied to front sides of Nose/Mouth opening of Cold Cap to receive Oxygen Mask

12. Velcro applied to top front neck flap to receive Oxygen Mask

13. Outside of neck collar

14. Neck collar

15. Neck opening

16. Vertical Coolant Cells all around perimeter of Cold Cap

17. Inner latex-like layer

18. Screw-on cap for Oxygen Mask

What is claimed as new and desired to be secured by Letters Patent of the United States is:

1. A head covering device comprised of:
A) An insulating material comprising cells filled with a non-lethal fluid kept well below freezing,
B) The device is used to cover the head of victims who appear to have expired from asphyxiation caused by reasons of prolonged oxygen deprivation,
C) This device, placed on the head of the victim, would slow, if not stop, the deterioration of the brain cells resulting from the cessation of the blood circulatory system,
D) Even though an individual appears to have expired he may be revived during a period of time before the brain, deprived of sufficient oxygen, actually does expire.

2. In accordance with claim 1:
A) Wherein the means to apply reduced temperature within the enclosure comprises fluid tight containment cells disposed to cover the interior walls of said enclosure with a fluid capable of receiving and retaining temperature cooling filling each of the said containment cells sufficient to achieve the arresting of damage caused by cessation of blood flow to the brain,
B). Also, in accordance with claim 1, the device would be stored in freezers located in areas available for immediate use to keep the circulatory blood system of the victim in a state of arrest, delaying brain damage or death,
C) These freezers can be designed and installed in any location,—not only the locations directed by the police, fire departments, medical facilities and other rescue type organizations, Also, residential type refrigerator freezers could be used to store the said Cold Cap devices,
D) It must be understood that the illustration described herein can be subject to change in regard to the size and type of compartment system and coolant and oxygen mask appliance, as well as the materials that are shown to make up the device.
E) This Cold Cap would give the rescuer an opportunity to get medical help for the victim, Time will be of the essence.

3. An additional claim is necessary to protect the rights of the inventor:
A) When a victim loses consciousness and the heart stops beating, and the victim is no longer breathing, there is a matter of only minutes before the victim is lost,
B) My theory is that with the said Cold Cap readily available and put into use, the clock stops, and the said Cold Cap extends the time available to save the life of the victim,
C) If the rescuers had the said Cold Cap with them when responding to emergencies such as burning buildings or drownings, victims could be saved by the Cold Cap while waiting for resuscitation, The Cold Cap should be applied immediately to the head of all apparently non-breathing victims; then the action to revive the victims can be started.
